(19) United States
(12) Patent Application Publication
(10) Pub. No.: US 2025/0356951 A1
(43) Pub. Date: Nov. 20, 2025

(54) GENOMICS ALIGNMENT PROBABILITY SCORE RESCALER

(71) Applicant: TwinStrand Biosciences, Inc., Seattle, WA (US)

(72) Inventors: Charles C. VALENTINE, III, Seattle, WA (US); Robert AZAD, Seattle, WA (US)

(21) Appl. No.: 18/867,339

(22) PCT Filed: May 19, 2023
(86) PCT No.: PCT/US2023/022945

§ 371 Date: Nov. 19, 2024

Related U.S. Application Data
(60) Provisional application No. 63/344,463, filed on May 20, 2022.

(51) Int. Cl. G16B 30/10; G16B 30/20

(52) U.S. Cl. CPC G16B 30/10; G16B 30/20

(57) ABSTRACT

Provided herein are methods and systems for aligning next-generation sequence reads to a reference genome such that genetic conditions or diseases, including rare variants or mutations, may be identified. Provided herein are methods of mapping a query sequence. In some aspects, the methods include receiving a set of alignments of a query to a reference sequence, each alignment in the set of alignments corresponding to the query aligning to a subsequence of the reference, wherein each alignment is assigned an initial mapping probability.

### CROSS-REFERENCE TO RELATED APPLICATIONS

This application claims the benefit of U.S. Provisional Appl. No. 63/344,463, filed May 20, 2022, the disclosure of which is incorporated by reference herein in its entirety.

### TECHNICAL FIELD

This disclosure relates generally to sequencing methodology that provides solutions for identifying and/or correcting errors in next generation sequencing (NGS) outputs such that rare variants or mutations may be identified.

### BACKGROUND

Genetics in general is a branch of biology concerned with the study of genes, genetic variation, and heredity in organisms. Genetic information is encoded in deoxyribonucleic acid (DNA) or ribonucleic acid (RNA) and is a succession of nucleotides or modified nucleotides representing the primary structure of nucleic acids. The genome refers to the complete set genetic material present in a cell or organism.

Duplex sequencing is a method for NGS platforms that employs tagging of DNA to detect mutations with higher accuracy and lower error rates. Additionally, duplex sequencing uses molecular tags and sequencing adapters to relate and distinguish reads originating from both strands of a DNA molecule and form a duplex consensus sequence from the two strands.

In existing duplex sequencing techniques, a problem arises for generating a duplex consensus sequence if reads from half of a duplex map to one genomic location on a reference genome but reads of the other half of the duplex map to a different location on the reference genome (as can be the case for genomic repeats, or genes or pseudogenes with very high percent identity). This can occur because conventional sequence aligners will typically select from multiple loci at random to set a primary alignment when a sequence read aligns equally well to the multiple loci within a reference sequence. Thus, during the step of aligning such sequence reads to a reference genome (e.g., for pairing related sequence reads, to identify sequence variations, etc.), conventional alignment processes can end up separating strand information into two different areas of the reference sequence, thereby losing track of the relatability of the strands for error correction and variant detection.

### SUMMARY

Provided herein are system, apparatus, article of manufacture, method and/or computer program product aspects, and/or combinations and sub-combinations thereof which provides solutions for identifying and/or correcting errors in next generation sequencing (NGS) outputs such that rare variants or mutations may be identified.

In some aspects, provided herein are methods of mapping a query sequence. In some aspects, the methods include receiving a set of alignments of a query to a reference sequence, each alignment in the set of alignments corresponding to the query aligning to a subsequence of the reference, wherein each alignment is assigned an initial mapping probability. The set of alignments may include an alignment that overlaps with a target subsequence of the reference sequence and an alignment that does not overlap with the target subsequence. The mapping probability of at least one alignment is then revised by applying at least one change. The change may increase the mapping probability for any of the alignments that overlap with the target subsequence, thereby producing a first revised mapping probability. Alternatively or additionally, the change may decrease the mapping probability for any of the alignments that do not overlap with the target subsequence, thereby producing a second revised mapping probability. A primary alignment (and optionally a secondary alignment) may be assigned based at least in part on the revising of at least one mapping probability. The primary alignment (and optionally the secondary alignment) is then output in an alignment output file.

In some aspects, receiving the set of alignments includes grouping the set of alignments by their read names. In some aspects, the method further includes repairing a sequence read or set of related sequence reads using the respective primary alignment. In some aspects, the primary alignment selected has a highest probability of accuracy out of all possible alignments.

In some aspects, revising the at least one mapping probability includes applying the first change and applying the second change.

In some aspects, selecting the primary alignment is further based on at least one of: sequence information in the query; the quality of one or more base calls in the query; one or more matches, mismatches, insertions or deletions at a position or region of interest within the alignment; clipping of the query, or a combination thereof.

In some aspects, the query is a sequence or a set of related sequences obtained from a biological sample.

In some aspects, the query comprises a DNA sequence or a set of related DNA sequences. In some aspects, the query comprises a sequencing read. In some aspects, the query comprises a set of related sequencing reads. In some aspects, the set of related sequencing reads comprises a set of paired end sequencing reads.

In some aspects, “overlap with the target subsequence” includes at least one read in the set of related sequencing reads overlapping with the target subsequence.

In some aspects, the receiving the set of alignments includes grouping the set of alignments by a name assigned to the read or a name assigned to the set of related sequencing reads.

In some aspects, the method further comprises repairing the read or at least one read of the set of related sequencing reads after applying the first change or applying the second change.

In some aspects, the method further comprises repairing the read or at least one read of the set of related sequencing reads after selecting the primary alignment.

In some aspects, selecting the primary alignment comprises comparing the mapping probability assigned to each alignment in the set of alignments and identifying the alignment having a highest mapping probability.

In some aspects, a highest probability of the initial mapping probabilities is not assigned to any of the alignments that overlap with the target subsequence.

In some aspects, the reference sequence comprises a reference genome assembly, a set of reference scaffolds, a set of reference contigs, or a set of reference reads or fragments.

In some aspects, the query comprises an output of whole genome sequencing, whole exome sequencing, or targeted sequencing that is enriched for a genomic region corresponding to the target subsequence of the reference sequence.

In some aspects, the target subsequence comprises a coding region, a non-coding region, or a combination thereof. In some aspects, the target subsequence comprises a nuclear DNA sequence or a mitochondrial DNA sequence. In some aspects, the target subsequence comprises a synthetic DNA sequence.

In some aspects, the target subsequence comprises a cancer-associated gene. In some aspects, the cancer-associated gene is selected from U2 Small Nuclear RNA Auxiliary Factor 1 (U2AF1) and Putative potassium voltage-gated channel subfamily E member 1B (KCNE1B).

In some aspects, the first change and/or the second change comprises a fold change.

In some aspects, the first change and/or the second change comprises a change determined by a Bayesian approach.

In some aspects, the initial mapping probabilities are considered priors.

In some aspects, the method further comprises:
identifying a set of coordinates of the reference sequence as a skippable region;
determining that the set of alignments includes an alignment having a highest mapping probability; and
determining that the alignment having the highest mapping probability does not correspond to the skippable region.

Also provided herein are methods of mapping a plurality of query sequences, comprising, for each of the query sequences of the plurality, mapping the query sequence by a method of mapping a query sequence as described herein.

Also provided herein are methods of mapping a duplex-stranded set of query sequences, comprising mapping a first query sequence by a method of mapping a query sequence as described herein;
mapping a second query sequence by a method of mapping a query sequence as described herein;
identifying the first query sequence and the second query sequence as each having a primary alignment that corresponds to a same set of genomic coordinates of the reference sequence; and
determining that the first query sequence and the second query sequence originate from complementary strands of a same original double-stranded template molecule;
wherein the first query sequence and the second query sequence each comprise a single molecule identifier (SMI) comprising coordinates of the primary alignment and a strand-defining element (SDE).

In some aspects, for at least one of the first query sequence and the second query sequence, a highest probability of the initial mapping probabilities is not assigned to any of the alignments that overlap with the target subsequence.

In some aspects, the method further comprises generating a duplex consensus sequence based on at least a subsequence of each of the first query sequence and the second query sequence.

In some aspects, the SMI comprises one or more coordinates of the primary alignments. In some aspects, the SMI comprises an exogenous sequence attached to the original template molecule, an endogenous sequence present on an end of the original template molecule, or a combination thereof.

Also provided herein are systems comprising: a processor; and a non-transitory computer readable medium containing instructions that, when executed by the processor, cause the processor to perform a method of mapping a query sequence as described herein, a method of mapping a plurality of query sequences as described herein, or a method of mapping a duplex-stranded set of query sequences as described herein.

Also provided herein are non-transitory computer readable storage media having computer readable instructions stored thereon that, when executed by a computer system, cause the computer system to perform a method of mapping a query sequence as described herein, a method of mapping a plurality of query sequences as described herein, or a method of mapping a duplex-stranded set of query sequences as described herein.

Further features of the present disclosure, as well as the structure and operation of various aspects, are described in detail below with reference to the accompanying drawings. It is noted that the present disclosure is not limited to the specific aspects described herein. Such aspects are presented herein for illustrative purposes only. Additional aspects will be apparent to persons skilled in the relevant art(s) based on the teachings contained herein.

### BRIEF DESCRIPTION OF THE DRAWINGS/FIGURES

The accompanying drawings, which are incorporated herein and form a part of the specification, illustrate aspects of the present disclosure and, together with the description, further serve to explain the principles of the disclosure and to enable a person skilled in the art(s) to make and use the aspects.

FIG. 1 illustrates a flowchart of an example sequencing method, according to an aspect of the disclosure.

FIG. 2 illustrates a flowchart of another example sequencing method, according to an aspect of the disclosure.

FIG. 3 depicts alignments generated using an example duplex sequencing method, according to an aspect of the disclosure.

FIG. 4 depicts an example computer system useful for implementing various aspects.

The features of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. Unless otherwise indicated, the drawings provided throughout the disclosure should not be interpreted as to-scale drawings.

### DETAILED DESCRIPTION

Aspects of the invention will now be described more fully hereinafter with reference to the accompanying drawings, in which aspects of the disclosure are shown. The aspects may, however, be embodied in many different forms and should not be construed as limited to the aspects set forth herein. Rather, these aspects are provided so that this disclosure will be thorough and complete, and will fully convey the scope of the disclosure to those skilled in the art.

Most genetic conditions or diseases have genetic signatures-a particular ordering of nucleotides in a gene that is responsible for the presence or absence of a genetic condition in the patient. In order to determine whether a patient has a particular genetic condition or disease, that patient's DNA may be analyzed for the presence or absence of the genetic signature. In some instances, a target sequence is a sequence of DNA that may include the genetic signature. As one example, the genetic signature is a cancer-related mutation, and the target sequence is a gene that may harbor a cancer-related mutation. A gene that may harbor a cancer-related mutation is referred to herein as a cancer-associated gene.

A locus is a specific, fixed position or set of positions on a chromosome where a particular gene or genetic marker is located. When two or more genomic loci contain sequences with a high percentage identity, the reduced sequence diversity in the reference sequence at the loci may cause a query sequence to align equally well to those loci. In this case, conventional sequence aligners often select one of these loci at random (e.g., a virtual coin-flip) and set the randomly chosen loci as the primary location for the sequence read alignment output and possibly setting all the other loci as secondary locations. This random selection can lead to miscataloging or loss of desired sequence information.

Such miscataloging of sequence information may be particularly problematic in duplex sequencing methods. While many sequencing mechanisms sequence only one strand of a DNA molecule, duplex sequencing tracks sequence information associated with both strands of an input double stranded DNA molecule. Duplex sequencing allows for building a consensus sequence using both strands of double-stranded DNA-if sequences obtained from both strands of the same original DNA molecule provide matching sequence information, then it is highly likely that the corresponding sequence reads were accurate (i.e., did not include errors introduced during sequencing). However, generating a duplex consensus can be difficult if reads from half of a duplex map to one reference location, while reads from the corresponding portion of the other half of the duplex map to a different reference location. This problem can occur for genomic repeats or genes (and pseudogenes) with a very high percentage identity.

Aspects of the present invention provide an improved way to select a primary sequencing alignment when multiple alignment possibilities exist for a particular queried sequence. Specifically, when mapping reads to a reference genome, aspects first determine whether a read has a primary alignment that does not overlap with a genomic region of interest (“ROI,” also referred to herein as a target subsequence), but there exists an alternative alignment that does overlap with the genomic ROI. If so, aspects of the invention boost (i.e., increase) an alignment probability score for the alternative alignment (in the genomic ROI). This boost may be a per target boost (i.e., a boost calculated separately for each target) or may be an equal boost for multiple or all targets of interest (for example, in a gene panel). The boost may be a fold-change in the probability. Additionally, or alternatively, aspects of the invention decrease an alignment probability score for a primary alignment that maps to a non-desired region, when an alternative alignment maps to a genomic ROI. This decrease may be a per region decrease (i.e., a decrease calculated separately for non-desired region) or may be an equal decrease for multiple or all non-desired regions. The decrease may be a fold-change in the probability. That is, there is a probability Pr associated with a specific genomic interval (i.e., target sequence) that represents the likelihood, or expectation, of the locus being observed over its multi-mapping counterparts. The value may be encoded as a ratio or fold-change, and may be used to scale certain values of read alignments that overlap with the input genomic intervals. New primary alignments may be selected based on the rescaled values, such that alignments with the greatest rescaled values are then set as the primary alignment. By rescaling a probability score to favor certain alignments overlapping with a target sequence of interest, likelihood of losing desired sequencing information is reduced. In some aspects of the invention, instead of a fold change in probability, a Bayesian analysis may be performed to determine the probability boost. Aspects of the present invention further improve the likelihood of duplex reads mapping together, so that a duplex consensus can be generated and the existence of a target sequence determined. This solution boosts the likelihood of reads from both strands mapping to a genomic region of interest. It is of note that while aspects of the invention are described herein in the context of duplex sequencing, and while alignment methods disclosed herein have particular usefulness with duplex sequencing, the invention is not limited to use with duplex sequencing. For example, the alignment methods and analyses disclosed herein may be useful to single-strand consensus sequencing as well.

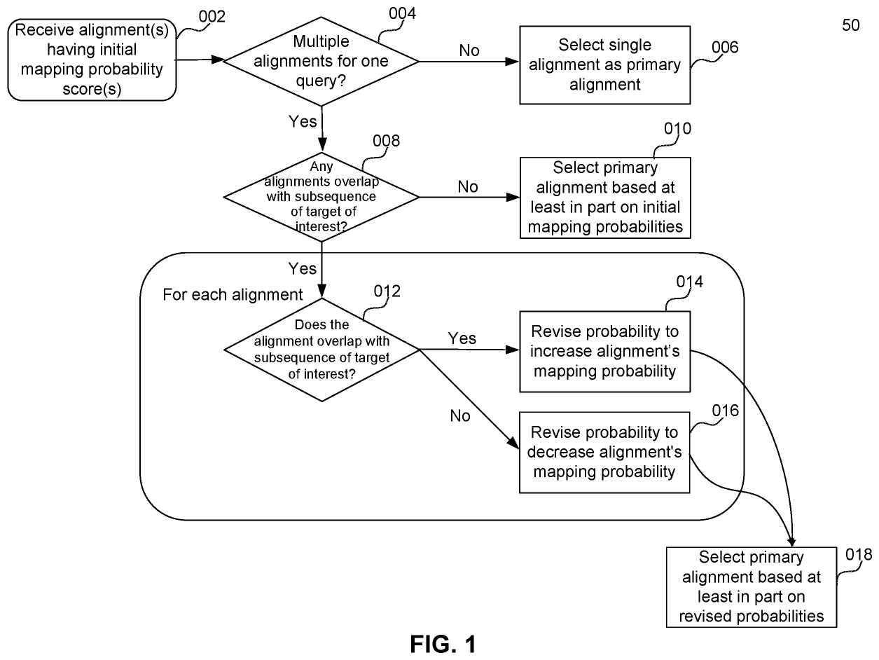

FIG. 1 depicts a flowchart describing a method 50 of computationally aligning sequences from an alignment query to a reference sequence, according to aspects of the invention. Method 50 outputs a primary alignment, and this method increases the likelihood of the output primary alignment overlapping with a target sequence of interest (also referred to herein as a target subsequence or ROI). In some aspects, the target sequence of interest is indicative of a particular genetic condition or disease. In some aspects, the alignment is output in an alignment output file, wherein the alignment output file comprises data stored on a non-transitory computer-readable storage medium (e.g., a SAM or BAM file, a text file, other file structure, or the like).

In step 002, a set of one or more alignments are received in response to a query. In some aspects, the query may request a return of all sequences and/or locations in a reference sequence that align with any of the queried sequence(s). A queried sequence is also referred to herein as a “query sequence.” The returned subsequences of a reference are referred to herein as alignments. In some aspects, the query includes a sequence or a set of related sequences obtained from a biological sample. In some aspects, the sequence(s) in the query include DNA sequence(s). In some aspects, the query includes a sequencing read or a set of related sequencing reads. In some aspects, the set of related sequencing reads includes a set of paired end sequencing reads. In some aspects, the query includes a consensus sequence, such as a consensus sequence generated from a set of reads.

In some aspects, the query includes an output of whole genome sequencing, whole exome sequencing, or targeted sequencing that is enriched for a genomic region corresponding to a target subsequence of a reference sequence. A target subsequence may be all or a portion of a full sequence of the target sequence of interest. In some aspects, the query includes an output of sequencing cell-free DNA.

Each alignment returned in response to a query may include or be assigned an initial mapping probability, which corresponds to the likelihood that the returned alignment is the correct alignment to the reference sequence for the queried sequence. In some aspects, the initial mapping probability may be provided as an initial mapping probability score. In some aspects, the initial mapping probability is considered to be a prior probability used in calculating a revised probability.

In some aspects, the reference sequence is a reference genome assembly, a set of reference scaffolds, a set of reference contigs, or a set of reference reads or fragments. In some embodiments, the reference sequence corresponds to a human genomic sequence.

In some aspects, where the query includes a sequencing read or set of related sequencing reads, receiving the set of alignments includes grouping the set of alignments by a name assigned to the read or a name assigned to the set of related sequencing reads. In some embodiments, the reads are obtained by a short-read sequencing method. Short-read sequencing methods, such as sequencing by synthesis or sequencing by ligation, may generate reads of approximately 75-400 nucleotides in length. Short reads are more prone to aligning to non-target sequences in a reference sequence as compared to long reads (e.g., as can be obtained by certain single-molecule sequencing techniques). In some embodiments, the reads are between 75 and 400 nucleotides in length.

In step 004, it is determined whether multiple alignments to the reference sequence exist for a given sequence or set of related sequences from the query. If only a single alignment is returned (e.g., there are not multiple alignments for the given sequence or set of related sequences), method 50 proceeds to step 006. In step 006, the single alignment returned in response to the query is selected as the primary alignment, which is then output.

If it is determined in step 004 that multiple alignments to the reference sequence exist for the queried sequence, method 50 proceeds to step 008.

In step 008, it is determined whether any of the returned alignments overlap with a subsequence of a target of interest. In some aspects, where the query includes a set of related sequences, the overlap with the target subsequence includes at least one read in the set of related sequences overlapping with the target subsequence. In some aspects, the target subsequence includes a coding region, a non-coding region, or a combination thereof. In some aspects, the target subsequence includes a nuclear DNA sequence or a mitochondrial DNA sequence. In some aspects, the target subsequence includes a synthetic DNA sequence.

In some embodiments, it is determined whether an initial primary alignment (i.e., an alignment having the highest initial mapping probability) overlaps with a preassigned region that does not overlap with the target sequence. In some aspects, when the initial primary alignment overlaps with a preassigned region that does not overlap with the target, then the revising includes applying a change to decrease the mapping probability for that alignment. For example, a region known to be a false duplication of a target sequence within a reference genome may be chosen as a preassigned region that does not overlap with the target. In particular embodiments, the preassigned region is a false duplication of U2AF1 within the hg18 reference sequence. In particular embodiments, the change to decrease the mapping probability for the alignment mapping to the preassigned region not overlapping with the target is applied, but a change to increase the mapping probability for the alignment mapping to the target is not applied.

In some aspects, the target subsequence (also referred to herein as a “target of interest” or “ROI”) comprises a coding region, a non-coding region, or a combination thereof. In some aspects, the target subsequence comprises a coding region. In some aspects, the target subsequence comprises a non-coding region. In some aspects, the target subsequence comprises a nuclear DNA sequence. In some aspects, the target subsequence comprises a mitochondrial DNA sequence. In some aspects, the target subsequence comprises a synthetic DNA sequence.

In some aspects, “overlapping with a target of interest” includes a padding region that extends beyond a genomic interval of a target of interest. For example, the padding region may extend 150 base pairs beyond a genomic interval of a target of interest. For example, the gene U2AF1 has a genomic interval within the hg38 human genome assembly of hg38 chr21: 43,092,956-43, 107,570. In this case, overlapping with the U2AF1 target includes any alignment interval starting or stopping within chr21: 43,092,806-43,107,720, which includes coordinates beginning 150 nucleotides before the start of the U2AF1 gene and ending 150 nucleotides after the end of the U2AF1 gene. Padding regions that extend beyond a target of interest may be useful, for example, when sequencing libraries are subjected to random fragmentation and hybrid selection. In these instances, some sequencing reads that belong to the target gene may partially overlap with the gene and extend beyond the target.

In some aspects, the target subsequence comprises a target nuclear DNA subsequence. In such aspect, the set of alignments may include an alignment that overlaps with the target nuclear DNA subsequence, and at least one of alignment that does not overlap with the target nuclear DNA subsequence but instead overlaps with a mitochondrial DNA subsequence. In some embodiments, the mapping probability for the alignment that overlaps with the target nuclear DNA subsequence is increased and/or the mapping probability for the at least one alignment that instead overlaps with a mitochondrial DNA subsequence is decreased. As an example of the utility of such aspects, there is a near identical ˜1 kb sequence within the nuclear and mitochondrial genomes of mouse strains. Using conventional alignment techniques, the probability of a read mapping to identical mitochondrial and nuclear locations is directly proportional to the ratio of mitochondrial genomes versus that of nuclear genomes. Thus, for a target of interest in a nuclear genome, increasing the mapping probability for alignments that overlap with the target nuclear DNA sequence and/or decreasing the mapping probability for alignments that instead overlap with the identical mitochondrial locations can help reduce the loss of desired sequence information.

In some aspects, the target subsequence comprises a target mitochondrial DNA subsequence. In such aspect, the set of alignments may include an alignment that overlaps with the target DNA mitochondrial subsequence, and at least one of alignment that does not overlap with the target mitochondrial DNA subsequence but instead overlaps with a nuclear DNA subsequence. In some embodiments, the mapping probability for the alignment that overlaps with the target mitochondrial DNA subsequence is increased and/or the mapping probability for the at least one alignment that instead overlaps with a nuclear DNA subsequence is decreased.

In some aspects, the target subsequence includes a cancer-associated gene. The cancer-associated gene may be, for example and without limitation, U2 Small Nuclear RNA Auxiliary Factor 1 (U2AF1) or Putative potassium voltage-gated channel subfamily E member 1B (KCNE1B).

If it is determined in step 008 that none of the returned alignments overlap with a subsequence of the target of interest, method 50 proceeds to step 010. In step 010, the primary alignment for a given sequence from the query is selected and output based at least in part on the initial mapping probabilities. If it is determined in step 008 that one or more of the returned alignments do overlap with the subsequence of the target of interest, method 50 proceeds to step 012.

Step 012 begins a sub-process to revise the mapping probabilities of each alignment in the set of returned alignments. In step 012, for each alignment in the set of returned alignments, it is determined whether that particular alignment overlaps with a subsequence of the target of interest. If the alignment does not overlap with a subsequence of the target of interest, method 50 proceeds to step 016. In step 016, a change is applied to the mapping probability for that alignment such that the mapping probability is decreased. If it is determined in step 012 that the particular alignment being analyzed does overlap with a subsequence of the target of interest, method 50 proceeds to step 014. In step 014, a change is applied to the mapping probability for that alignment such that the mapping probability is increased.

In some aspects, the change of step 014 and/or the change of step 016 includes a fold change. In some aspects, the change of step 014 and/or the change of step 016 includes a change determined by a Bayesian approach.

In some aspects, the change of step 014 and/or the change of step 016 includes a change that is proportional to a ratio of the copy number of a subsequence of a target of interest versus the copy number of a duplicate sequence elsewhere in the genome. For example, for a mitochondrial genome target of interest having one duplication within the nuclear genome, if the copy number of the mitochondrial genome target of interest is two (e.g., if there are two copies of the mitochondrial genome present), a 2-fold increase in probability could be chosen for the mitochondrial gene to weight the mapping probabilities based on copy number.

Once the mapping probability for each alignment returned from the query is revised according to steps 014 and 016, method 50 proceeds to step 018. In step 018, a primary alignment for the queried sequence is selected based at least in part on the revised mapping probability (ies). Depending on the number of alignments returned in response to a given query, steps 014 and 016 may both be performed in order to select a primary alignment in response to the given query. In some aspects, selecting the primary alignment includes comparing the mapping probability assigned to each alignment in the set of alignments, and identifying the alignment having the highest mapping probability. In some aspects, selecting the primary alignment is further based on at least one of: sequence information in the query; the quality of one or more base calls in the query; one or more matches, mismatches, insertions, or deletions at a position or region of interest within the alignment; clipping of the query; or a combination thereof.

In some aspects, none of the initial mapping probabilities for the alignment(s) that overlap with the target subsequence had the highest probability in the initial mapping probabilities.

In some aspects, where the query includes a read or a set of related reads, the read or at least one read in the set of related reads is repaired. In some aspects, the read or at least one read in the set of related reads is repaired after applying the change to increase the mapping probability score and/or after applying the change to decrease the mapping probability score. In some aspects, the read or at least one read in the set of related reads is repaired after the primary alignment is selected. In some embodiments, the repairing is necessary because the aligner saves disc space by only including certain information about the read in the primary alignment (not the secondary alignment(s)). Thus, repairing the read can involve transferring or porting over sequence read information to the new primary such that the new primary has inherited the information. In instances where the data from the initial primary is not available, dummy data or missing data markers may be used.

In some aspects, method 50 may be performed for each of a plurality of query sequences, and include mapping the plurality of query sequences based on the primary alignment output for each sequence in the plurality of query sequences. For example, the plurality of query sequences may include a duplex-stranded set of query sequences. In some aspects, method 50 is performed for each of a first query sequence and a second query sequence. Each of the first query sequence and the second query sequence may be identified as having a primary alignment that corresponds to a same set of genomic coordinates of the reference sequence. It may then be determined that the first query sequence and the second query sequence originate from complementary strands of a same original double-stranded template model. In some aspects, method 50 further includes generating a duplex consensus sequence based on at least a subsequence of the first query sequence and the second query sequence.

In some aspects, the first query sequence and the second query sequence are each associated with a single molecule identifier (SMI) and a strand-defining element (SDE). In some aspects, the SMI includes sequence that maps to the primary alignment. In some aspects, the SMI includes an exogenous sequence attached to the original template molecule, an endogenous sequence present on an end of the original template molecule, or a combination thereof. In some aspects, for at least one of the first query sequence and the second query sequence, a highest probability of the initial mapping probabilities was not assigned to any of the alignments that overlap with the target subsequence.

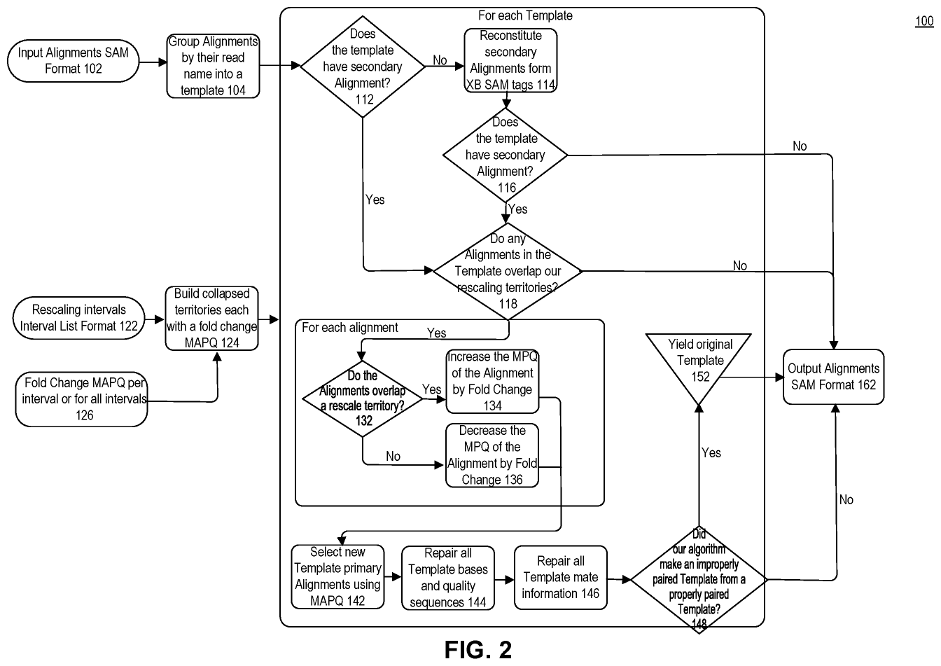

FIG. 2 depicts a flowchart describing a method 100 of computationally aligning sequences from a DNA strand by a sequencing aligner, according to aspects of the invention. Method 200 is one example implementation of method 50, although method 50 is not limited thereto.

In step 102, a set of initial alignments returned in response to a query are input into an aligner configured in accordance with aspects of the present invention. The initial alignments may include alignments corresponding to each strand of a duplex DNA molecule. The initial alignments may have been generated by any known alignment method that maps sequence reads from a query to a reference genome. The sequence reads may have been generated using any known sequencing technique, such as any next-generation sequencing technique. These initial alignments may be output, for example, as one or more records in a sequence alignment map (SAM) format. The aligner can then include any secondary information (such as secondary alignments) in the metadata associated with the SAM record output. In some embodiments, the aligner is the Burrow-Wheeler Aligner (BWA).

SAM is a text-based format that can store biological sequences that are aligned to the reference sequence. The SAM format has a header and an alignment section with a variety of fields, as referenced in Table 1. The fields listed in Table 1 are always present (i.e. mandatory) in the alignment section, and more may be present as well.

TABLE 1

| Field | Type | Description |
|---|---|---|
| QNAME | String | Query template name |
| FLAG | Int | Bitwise FLAG |
| RNAME | String | References sequence NAME |
| POS | Int | 1 based leftmost mapping POSition |
| MAPQ | Int | MAPping Quality |
| CIGAR | String | Cigar String |
| RNEXT | String | Ref name of the mate/next read |
| PNEXT | Int | Position of the mate/next read |
| TLEN | Int | Observed Template Length |
| SEQ | String | Segment sequence |
| QUAL | String | ASCII or Phred-scaled based Quality + 33 |

The probability that the alignment was incorrect (e.g., the initial mapping probability) is typically encoded in the MAPQ SAM field. The MAPQ SAM field refers to the mapping quality. For example, MAPQ may equal to −10 log₁₀ Pr {mapping position is wrong}, rounded to the nearest integer. In aspects of the present invention, it is this MAPQ value that is rescaled to determine a final primary alignment.

Once the set of initial alignments are input into the aligner in step 102, method 100 proceeds to step 104. In step 104, the alignments in the set of alignments are grouped and put into a template. In some aspects, the alignments are grouped according to their read name. When grouping, the read name and position, for example, can be used. A group alignment pair describes how a single read pair was aligned.

Once the alignments are grouped and put into the template in step 104, method 100 proceeds to step 112. In step 112 it is decided whether the template has secondary alignments or not. If the template has secondary alignments, this means there is not complete consensus as to what the correct alignment should be. In this case, the method moves on to step 118. If the template does not have secondary alignments, then method 100 moves on to step 114.

In step 114 the process reconstitutes secondary alignments from a SAM tag key (e.g., a XB SAM tag), and method 100 proceeds to step 116. “XB SAM tag” refers to a key used by the BWA aligner, which can hold the auxiliary data for supplementary alignments (such as secondary alignments) along with their alignment score. For an aligner that does not by default include a tag with a secondary alignment score, a user-defined tag may be modified to include this information.

In step 116, the process decides again if the template has secondary alignments. If secondary alignments are present, which may indicate that consensus has not been reached, the process moves on to step 118. If the template still has no secondary alignments after step 114, then consensus has likely been reached and the process proceeds to step 162, outputting the alignments. In some aspects, this corresponds to step 006 in FIG. 1. In some aspects, the alignments output in step 162 are in the SAM format.

In step 118, where the template alignments (i.e., the set of alignments) contain at least both primary and secondary alignments, it is determined whether any alignments in the template overlap rescaling territories. A rescaling territory, also referred to herein as a subsequence of a target of interest or a target subsequence, is a portion of a sequence that includes all or a portion of the target sequence of interest. In some aspects, the rescaling territory can include a padding region of 150 nucleotides extending beyond a genomic interval for a target sequence of interest. Determining whether an alignment overlaps a rescaling territory involves determining whether all or a part of a nucleotide sequence corresponding to an individual alignment is the same as all or a part of a nucleotide sequence corresponding to the target sequence of interest. In some aspects, it is determined whether the entirety of a sequence read being aligned is contained within the target sequence of interest. In some aspects, it is determined whether a portion of the sequence read being aligned corresponds to all or a portion of the target sequence of interest.

If it is determined in step 118 that the alignments in the template do not overlap any rescaling territories, method 100 moves to step 162 and outputs the alignments. In some aspects, this corresponds to step 010 in FIG. 1. The process is finalized as there is nothing to rescale or repair. If it is determined in step 118 that the alignments in the template do overlap the rescaling territories, method 100 moves on to step 132.

While steps 112-118 evaluated the template of alignments as a whole, steps 132-136 are performed for each alignment in the template of alignments. In step 132, for each alignment, it is determined whether or not that specific alignment overlaps a rescale territory. In some aspects, this corresponds to step 012 in FIG. 1. The identification of rescaled territories is illustrated in steps 122-126, according to an aspect. In step 122, an interval list is provided by the user. An interval list defines genomic regions of interest, which may include subsets of regions and/or individual positions in the genome. In aspects, the interval list may include one or more target sequences of interest. The interval list may be provided in, for example, interval list format. An interval list can be provided in a similar format to a bait and target interval list (as used for hybrid capture indicating the genomic intervals targeted by bait/probes). It is recommended that a sequence dictionary of the interval list should match that of the reference genome used for alignment.

In some aspects, the methods of mapping a query sequence as described herein include determining if the template alignments or set of alignments includes an alignment that overlaps with a skippable region. Skippable regions as referred to herein do not overlap with a target sequence and are a set of coordinates within a reference sequence for which the rescaling or revising of the mapping probabilities is not desired. A skippable region may be desirable, for example, when a reference sequence includes one “true” copy or a target sequence, a false duplication of the target sequence, and a pseudogene or other segment of the genome sharing a high level of identity (such at least 80%, at least 85%, at least 90%, at least 95%, at least 98%, or at least 99% identity, or a secondary alignment is produced using an alternative scoring strategy that is not strictly based on the identity of aligned and reference sequences) with the target sequence. In some embodiments, when an alignment having the highest initial mapping probability is determined as overlapping with a skippable region, then the revising of the initial mapping probabilities is not performed for that set of alignments.

In step 126, one or more rescaling values corresponding to the intervals in the interval list are provided. In some aspects, each interval in the interval list is assigned its own rescaling value. A rescaling value is also referred to herein as a change applied to an initial mapping probability. In some aspects, a single rescaling value applies to multiple intervals in the interval list. In some aspects, a single rescaling value applies to all intervals in the interval list. In some aspects, the rescaling value is a fold change MAPQ value. In some embodiments, the rescaling value is determined using a Bayesian analysis.

In step 124, the interval list from step 122 and the rescaling values from step 126 are combined to generate a collapsed list of rescaling territories, wherein each rescaling territory is associated with a respective rescaling value.

Returning to step 132, each alignment in the template received from step 118 is compared to the rescaling territories identified in step 124 to determine whether the alignment overlaps a rescale territory.

For each alignment, if the alignment overlaps a rescale territory, then method 100 proceeds to step 134. In step 134, the probability value corresponding to that alignment, such as the MAPQ value, is rescaled by increasing the probability by the rescaling value identified in the collapsed list from step 124. In some aspects, this corresponds to step 014 in FIG. 1. If it is determined in step 132 that the alignment does not overlap a rescale territory, then method 100 proceeds to step 136. In step 136, the probability value corresponding to that alignment, such as the MAPQ value, is rescaled by decreasing the probability value according to a rescaling value. In some aspects, this corresponds to step 016 in FIG. 1. In some aspects, the probability value may be decreased by a default rescaling value (such as inverting the rescaling value applied in step 134), or by a rescaling value identified in the collapsed list from step 136.

Once method 100 completes either step 134 or step 136 for all alignments in the template of alignments, method 100 proceeds to step 142. In step 142, the rescaled probability values (e.g., MAPQ values) are used to at least in part to select new template primary alignments. In some aspects, another quality additionally used to select a primary alignment is whether the alignment is properly oriented (with favor given to alignments that are properly oriented rather than improperly oriented). Because of the rescaling, the primary alignments identified in step 142 are statistically the most accurate. In some aspects, the new template primary alignments include at least one alignment whose initial MAPQ value was not the highest out of all the initial alignments. In some aspects, the new template primary alignments also include at least one alignment whose initial MAPQ value was the highest out of all the initial alignments.

After selecting new template primary alignments in step 142, method 100 proceeds to step 144. In step 144, all template bases and quality sequences are repaired. This may involve, for example, correcting an overall DNA sequence based on the primary alignment selected for a particular sequence read in step 142.

Method 100 then proceeds to step 146. In step 146, all template mate information is repaired (e.g., for a duplex-stranded set of sequences). After repairing all template mate information, method 100 proceeds to step 148. In step 148, it is determined whether or not the process made an improper paired template from a properly paired template. A properly paired template may be one where sequence reads on corresponding locations of each of a pair of DNA strands match. An improperly paired template may be one where sequence reads on corresponding locations of each of a pair of DNA strands do not match. If the template did make an improperly paired template, then method 100 proceeds to step 152, where the original template is yielded. If the template did not make an improperly paired template, then method 100 proceeds to step 162. In step 162, the process is finalized and the alignments are output. The output may be, for example, a SAM file with the alignments provided in SAM format.

This rescaling of MAPQs and primary selection is performed per template on primary and secondary alignments. All supplementary alignments are included in the output but are not considered for rescaling. The method described in FIG. 2 biases alignments to a target genomic interval of a reference sequence if that reference sequence also contains a duplicated or near identical sequence elsewhere.

The flowchart of FIG. 2 can be used for a variety of genes. For one illustrative example, the U2AF1 gene, which is a protein coding gene, may be targeted in an assay. An artifact 760 bp segmental duplication (i.e. identical sequence) of the U2AF1 gene exists at a second loci in the genome assembly.

Because of this artifactual duplication of the U2AF1 gene, an aligner may split alignments over the two duplicate loci such that raw coverage is randomly diluted by half at the U2AF1 target locus. In implementing method 100 as described above, a rescale mode may be used where an increasing change (e.g., an arbitrarily large fold-change value such as 1,000,000 or a prior probability of 100%) is supplied for the target locus. This results in all the of the primary artifact duplication alignments being reassigned to the targeted U2AF1 gene. By repeating this, a greater number of duplex consensus reads outputted at the target gene is yielded. Accordingly, more duplex consensus sequences may be generated and the sensitivity of detecting variants is increased.

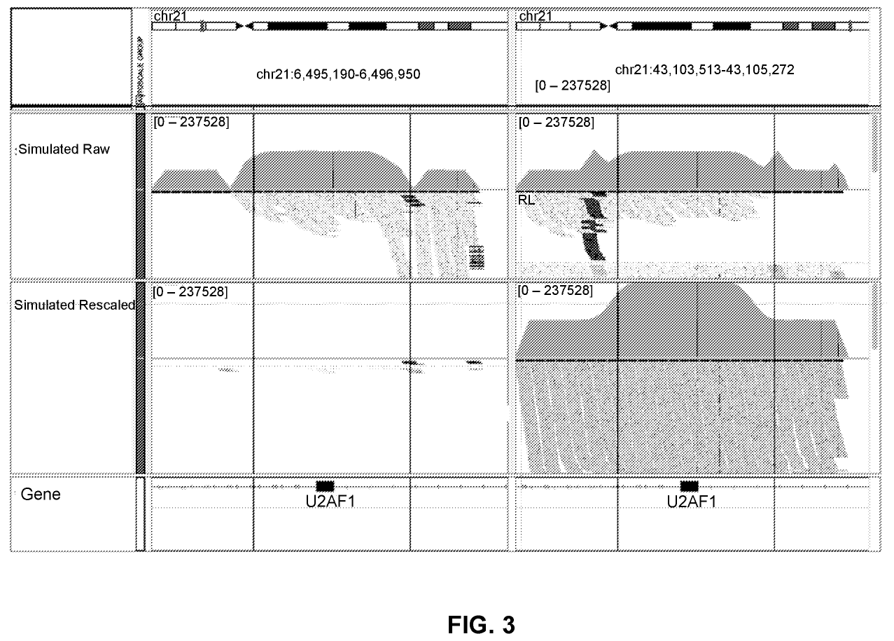

FIG. 3 depicts a histogram of relocated reads resulting from the simulated application of an aspect of method 100 to a sequence analysis using one exon of the U2AF1 gene as a target. The left half of the histogram shows a duplication of the U2AF1 gene that is present in the hg38 human genome assembly. The right side shows the actual U2AF1 gene. The top row depicts the simulated raw data of both the duplication of the U2AF1 gene and the actual U2AF1 gene. The middle row depicts the simulated rescaled data of both the duplication of U2AF1 and the actual U2AF1 gene. The bottom row depicts the gene being targeted, here U2AF1.

FIG. 3 further illustrates that a majority of alignments that initially assigned to the duplication are reassigned to the target U2AF1 gene.

In certain embodiments, a method as described herein can include one or more skippable regions, as previously described. In certain embodiments, a method for rescaling reads aligning to U2AF1 includes a U2AF1 pseudogene as a skippable region, such that when a region overlapping with the pseudogene is designated the initial primary alignment but a region overlapping with the U2AF1 gene is a secondary alignment, the rescaling (i.e., revising of the at least one mapping probability) is not performed. In particular embodiments, the skippable region of U2AF1 is designated as 15:35092812-35092971 (hg38).

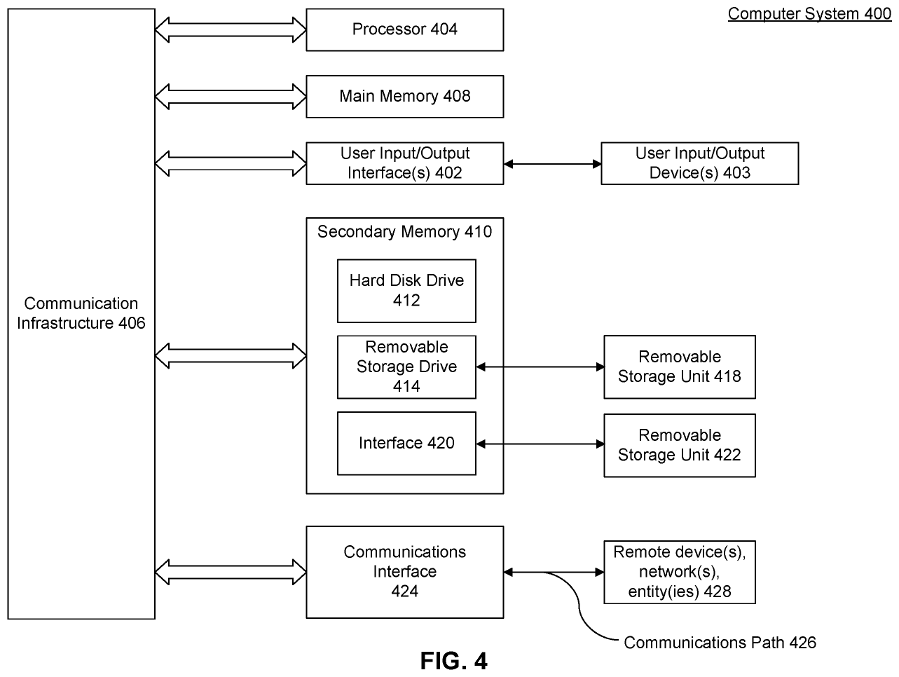

Various aspects may be implemented, for example, using one or more computer systems, such as computer system 400 shown in FIG. 4. One or more computer systems 400 may be used, for example, to implement any of the aspects discussed herein, as well as combinations and sub-combinations thereof. For example, one or more computer systems 400 may be used to execute method 50 and/or method 100 and display or otherwise provide the sequence alignment information output from method 50 and/or method 100. In some aspects, method 50 and/or method 100 are encoded as instructions on a non-transitory computer readable storage medium that, when executed by computer system 400, causes computer system 400 to perform the steps of method 50 and/or method 100, respectively.

Computer system 400 may include one or more processors (also called central processing units, or CPUs), such as a processor 404. Processor 404 may be connected to a communication infrastructure or bus 406. Processor 404 may be responsible for executing the instructions corresponding to method 50 and/or method 100.

Computer system 400 may also include user input/output device(s) 402, such as monitors, keyboards, pointing devices, etc., which may communicate with communication infrastructure 406 through user input/output interface(s) 402.

One or more of processors 404 may be a graphics processing unit (GPU). In an aspect, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 400 may also include a main or primary memory 408, such as random-access memory (RAM). Main memory 408 may include one or more levels of cache. Main memory 408 may have stored therein control logic (i.e., computer software) and/or data.

Computer system 400 may also include one or more secondary storage devices or memory 410. Secondary memory 410 may include, for example, a hard disk drive 412 and/or a removable storage device or drive 414. Removable storage drive 414 may be a floppy disk drive, a magnetic tape drive, a compact disk drive, an optical storage device, tape backup device, and/or any other storage device/drive.

Removable storage drive 414 may interact with a removable storage unit 418. Removable storage unit 418 may include a computer usable or readable storage device having stored thereon computer software (control logic) and/or data. Removable storage unit 418 may be a floppy disk, magnetic tape, compact disk, DVD, optical storage disk, and/any other computer data storage device. Removable storage drive 414 may read from and/or write to removable storage unit 418.

Secondary memory 410 may include other means, devices, components, instrumentalities or other approaches for allowing computer programs and/or other instructions and/or data to be accessed by computer system 400. Such means, devices, components, instrumentalities or other approaches may include, for example, a removable storage unit 422 and an interface 420. Examples of the removable storage unit 422 and the interface 420 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 400 may further include a communication or network interface 424. Communication interface 424 may enable computer system 400 to communicate and interact with any combination of external devices, external networks, external entities, etc. (individually and collectively referenced by reference number 428). For example, communication interface 424 may allow computer system 400 to communicate with external or remote devices 428 over communications path 426, which may be wired and/or wireless (or a combination thereof), and which may include any combination of LANs, WANs, the Internet, etc. Control logic and/or data may be transmitted to and from computer system 400 via communication path 426.

Computer system 400 may also be any of a personal digital assistant (PDA), desktop workstation, laptop or notebook computer, netbook, tablet, smart phone, smart watch or other wearable, appliance, part of the Internet-of-Things, and/or embedded system, to name a few non-limiting examples, or any combination thereof.

Computer system 400 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 300 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some aspects, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 400, main memory 408, secondary memory 410, and removable storage units 418 and 422, as well as tangible articles of manufacture embodying any combination of the foregoing. Such control logic, when executed by one or more data processing devices (such as computer system 400), may cause such data processing devices to operate as described herein. For example, the instructions, when executed by one or more processors, cause the processors to perform a method for managing third party applications on a computing apparatus as described herein.

Based on the teachings contained in this disclosure, it will be apparent to persons skilled in the relevant art(s) how to make and use aspects of this disclosure using data processing devices, computer systems and/or computer architectures other than that shown in FIG. 4. In particular, aspects can operate with software, hardware, and/or operating system aspects other than those described herein.

It is to be appreciated that the Detailed Description section, and not any other section, is intended to be used to interpret the claims. Other sections may set forth one or more but not all exemplary aspects as contemplated by the inventor(s), and thus, are not intended to limit this disclosure or the appended claims in any way.

While this disclosure describes exemplary aspects for exemplary fields and applications, it should be understood that the disclosure is not limited thereto. Other aspects and modifications thereto are possible, and are within the scope and spirit of this disclosure. For example, and without limiting the generality of this paragraph, aspects are not limited to the software, hardware, firmware, and/or entities illustrated in the figures and/or described herein. Further, aspects (whether or not explicitly described herein) have significant utility to fields and applications beyond the examples described herein. Aspects have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative aspects may perform functional blocks, steps, operations, methods, etc. using orderings different from those described herein.

References herein to “one aspect,” “an aspect,” “an example aspect,” “some aspects,” or similar phrases, indicate that the aspects described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it would be within the knowledge of persons skilled in the relevant art(s) to incorporate such feature, structure, or characteristic into other aspects whether or not explicitly mentioned or described herein.

Additionally, some aspects may be described using the expression “coupled” and “connected” along with their derivatives. These terms are not necessarily intended as synonyms for each other. For example, some aspects may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

The breadth and scope of this disclosure should not be limited by any of the above-described exemplary aspects, but should be defined only in accordance with the following claims and their equivalents.

1. A method of mapping a query sequence comprising:
receiving a set of alignments of a query to a reference sequence, each alignment in the set of alignments corresponding to the query aligning to a subsequence of the reference, wherein each alignment is assigned an initial mapping probability;
determining that the set of alignments comprises: (i) an alignment that overlaps with a target subsequence of the reference sequence and (ii) an alignment that does not overlap with the target subsequence;
revising at least one mapping probability comprising at least one of:
(i) applying a first change to increase the mapping probability for any of the alignments that overlap with the target subsequence, thereby producing a first revised mapping probability, and
(ii) applying a second change to decrease the mapping probability for at least a subset of the alignments that do not overlap with the target subsequence, thereby producing a second revised mapping probability;
selecting a primary alignment based at least in part on the revising at least one mapping probability; and
outputting the primary alignment in an alignment output file.

2. The method according to claim 1, wherein revising the at least one mapping probability comprises applying the first change and applying the second change.

3. The method of claim 1, wherein selecting the primary alignment is further based on at least one of: sequence information in the query; the quality of one or more base calls in the query; one or more matches, mismatches, insertions or deletions at a position or region of interest within the alignment; clipping of the query, or a combination thereof.

4. The method of claim 1, wherein the query is a sequence or a set of related sequences obtained from a biological sample, the query comprising at least one of:
a DNA sequence,
a set of related DNA sequences,
a sequencing read, or
a set of related sequencing reads.

5. (canceled)

6. (canceled)

7. (canceled)

8. The method of claim 4, wherein the set of related sequencing reads comprises a set of paired end sequencing reads.

9. The method of claim 4, wherein the overlap with the target subsequence comprises at least one read in the set of related sequencing reads overlapping with the target subsequence.

10. The method of claim 4, wherein the receiving the set of alignments includes grouping the set of alignments by a name assigned to the read or a name assigned to the set of related sequencing reads.

11. The method of claim 4, wherein the method further comprises at least one of:
repairing the read or at least one read of the set of related sequencing reads after applying the first change or applying the second change, or
repairing the read or at least one read of the set of the related sequencing reads after selecting the primary alignment.

12. (canceled)

13. The method of claim 1, wherein selecting the primary alignment comprises comparing the mapping probability assigned to each alignment in the set of alignments and identifying the alignment having a highest mapping probability.

14. The method of claim 1, wherein a highest probability of the initial mapping probabilities is not assigned to any of the alignments that overlap with the target subsequence.

15. The method of claim 1, wherein the reference sequence comprises a reference genome assembly, a set of reference scaffolds, a set of reference contigs, or a set of reference reads or fragments.

16. The method of claim 1, wherein the query comprises an output of whole genome sequencing, whole exome sequencing, or targeted sequencing that is enriched for a genomic region corresponding to the target subsequence of the reference sequence.

17. The method of claim 1, wherein the target subsequence comprises at least one of:
a coding region, a non-coding region, or a combination thereof,
a nuclear DNA sequence,
a mitochondrial DNA sequence,
a synthetic DNA sequence, or
a cancer-associated gene.

18. (canceled)

19. (canceled)

20. (canceled)

21. (canceled)

22. The method of claim 1, wherein the first change and/or the second change comprises at least one of a fold change or a change determined by a Bayesian approach.

23. (canceled)

24. (canceled)

25. The method of claim 1, wherein the method further comprises:
identifying a set of coordinates of the reference sequence as a skippable region;
determining that the set of alignments includes an alignment having a highest mapping probability; and
determining that the alignment having the highest mapping probability does not correspond to the skippable region.

26. (canceled)

27. (canceled)

28. A method of mapping a duplex-stranded set of query sequences, comprising
mapping a first query sequence by the method of claim 1;
mapping a second query sequence by the method of claim 1;
identifying the first query sequence and the second query sequence as each having a primary alignment that corresponds to a same set of genomic coordinates of the reference sequence; and
determining that the first query sequence and the second query sequence originate from complementary strands of a same original double-stranded template molecule;
wherein the first query sequence and the second query sequence each comprise a single molecule identifier (SMI) comprising coordinates of the primary alignment and a strand-defining element (SDE).

29. The method of claim 28 wherein for at least one of the first query sequence and the second query sequence, a highest probability of the initial mapping probabilities is not assigned to any of the alignments that overlap with the target subsequence.

30. The method of claim 28, wherein the method further comprises generating a duplex consensus sequence based on at least a subsequence of each of the first query sequence and the second query sequence.

31. The method of claim 28, wherein the SMI comprises at least one of:
one or more coordinates of the primary alignments, or
an exogenous sequence attached to the original template molecule, an endogenous sequence present on an end of the original template molecule, or a combination thereof.

32. (canceled)

33. A system comprising:
a processor; and
a non-transitory computer readable medium containing instructions that, when executed by the processor, cause the processor to perform the method of claim 1.

34. (canceled)
